(19) United States
(12) Patent Application Publication
(10) Pub. No.: US 2003/0225443 A1
(43) Pub. Date: Dec. 4, 2003

(54) Methods and devices for modulating atrial configuration

(76) Inventors: Kanthi Kiran, Modesto, CA (US); James M. Lovette, Palo Alto, CA (US); Jan B. Pietzsch, Stanford, CA (US); Nicholas J. Mourlas, Mountain View, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/388,606

(22) Filed: Mar. 13, 2003

Related U.S. Application Data
(60) Provisional application No. 60/364,165, filed on Mar. 13, 2002.
(60) Provisional application No. 60/384,633, filed on May 30, 2002.

(51) Int. Cl. A61N 1/00

(52) U.S. Cl. 607/119

(57) ABSTRACT

Methods and devices are provided for modulating atrial configuration, e.g., changing the configuration of an atrium, for example by reducing the volume of a left or right atrium. In practicing the subject methods, the configuration of an atrium is modified or changed at least partially without the use of an implant, e.g., through chemical modification and/or application of energy to atrial tissue, where representative energy sources include RF, microwave, laser, ultrasound, cryoablative energy sources, etc. In certain embodiments, the desired atrial configuration modification is achieved by reduction of the atrial volume, e.g., through reduction of the volume of, or constricting/closing the entrance to, the atrial appendage thereof, in a manner sufficient to reduce the volume of the atrium. In certain embodiments, a catheter device comprising an RF source is employed to modulate atrial configuration according to the subject methods. Also provided are devices, systems and kits for use in practicing the subject methods. The subject methods, devices, systems and kits find use in a variety of applications, including reducing the risk of stroke in a subject suffering from atrial fibrillation.

### INTRODUCTION

1. Field of the Invention

The field of this invention is embolic stroke and the treatment/prevention thereof.

2. Background of the Invention

Embolic stroke is the nation's third leading killer for adults, and is a major cause of disability. There are over 700,000 strokes per year in the United States alone. Of these, roughly 100,000 are hemorrhagic, and 600,000 are ischemic (either due to vessel narrowing or to embolism). The most common cause of embolic stroke emanating from the heart is thrombus formation due to atrial fibrillation. Approximately 80,000 strokes per year are attributable to atrial fibrillation.

Atrial fibrillation is an arrhythmia of the heart that results in a rapid and chaotic heartbeat that produces lower cardiac output and irregular, turbulent and/or stagnant blood flow in the vascular system. There are over five million people worldwide with atrial fibrillation, with about four hundred thousand new cases reported each year. Atrial fibrillation is associated with a 500 percent greater risk of stroke due to the condition. A patient with atrial fibrillation typically has a significantly decreased quality of life due, in part, to the fear of a stroke, and the pharmaceutical regimen necessary to reduce that risk.

For patients who develop atrial thrombus from atrial fibrillation, the clot normally occurs in the left atrial appendage (LAA) of the heart. The LAA is a cavity which looks like a small finger or windsock and which is connected to the lateral wall of the left atrium between the mitral valve and the root of the left pulmonary vein. The LAA normally contracts with the rest of the left atrium during a normal heart cycle, thus keeping blood from becoming stagnant therein, but often fails to contract with any vigor in patients experiencing atrial fibrillation due to the discoordinate electrical signals associated with AF. As a result, thrombus formation is predisposed to form in the stagnant blood within the LAA. Elimination or containment of thrombus formed within the LAA of patients with atrial fibrillation reduces the incidence of stroke.

Pharmacological therapies for stroke prevention such as oral or systemic administration of warfarin or the like have been inadequate due to serious side effects of the medications and lack of patient compliance in taking the medication. Invasive surgical or thorascopic techniques have been used to obliterate the LAA, however, many patients are not suitable candidates for such surgical procedures because of a compromised condition or having previously undergone cardiac surgery. In addition, the morbidity and potential risks of even a thorascopic surgical procedure often outweigh the potential benefits.

Despite the various efforts in the prior art, there remains a need for a minimally invasive method and associated devices for reducing the risk of thrombus formation in the left atrial appendage.

### SUMMARY OF THE INVENTION

Methods and devices are provided for modulating atrial configuration, e.g., changing the configuration of the atrium, for example by reducing the volume of a left atrium. In practicing the subject methods, the configuration of an atrium is modified or changed at least partially without the use of an implant, e.g., through chemical modification and/or application of energy to atrial tissue, where representative energy sources include RF, microwave, laser, ultrasound, cryoablative energy sources, etc. In certain embodiments, the desired atrial configuration modification is achieved by reduction of the atrial volume, e.g., through reduction of the volume of, or constricting/closing the entrance to, the atrial appendage thereof, in a manner sufficient to reduce the volume of the atrium. In certain embodiments, a catheter device comprising an RF source is employed to modulate atrial configuration according to the subject methods. Also provided are devices, systems and kits for use in practicing the subject methods. The subject methods, devices, systems and kits find use in a variety of applications, including reducing the risk of stroke in a subject suffering from atrial fibrillation.

### BRIEF DESCRIPTION OF THE FIGURES

FIG. 1 shows the catheter tip of a device according to an embodiment of the present invention inside the left atrial appendage during practice of a representative embodiment of the subject methods.

FIG. 2A shows a schematic view of a patient's left atrial appendage with an embodiment having features of the invention with a mechanically-deployed filtration element (filtration element closed).

FIG. 2B shows a schematic view of a patient's left atrial appendage with an embodiment having features of the invention with a mechanically-deployed filtration element (filtration element opened).

FIG. 3 shows a schematic view of a patient's left atrial appendage with an embodiment having features of the invention with an ambient shielding member.

FIG. 4 shows a schematic view of a patient's left atrial appendage with an embodiment having features of the invention with polar positioning capability.

FIG. 5 shows a schematic view of a patient's left atrial appendage with an embodiment having features of the invention with a splay tip element at the distal end of the catheter.

FIG. 6 shows a schematic view of a patient's left atrial appendage with an embodiment having features of the invention, where the embodiment is disposed within the appendage from within the heart.

FIG. 7 shows a schematic view of a patient's left atrial appendage with an embodiment having features of the invention, where the embodiment is positioned on the exterior of the appendage (transthoracical/transpericardial access).

FIGS. 8A, 8B and 8C show three different electrode configurations for a distal tip of a device according to the present invention.

FIG. 9 shows a catheter design with an occlusion balloon.

FIGS. 10A and 10B show a representative system according to the subject invention.

### DESCRIPTION OF THE SPECIFIC EMBODIMENTS

Methods and devices are provided for modulating atrial configuration, e.g., changing the configuration of the left atrium, for example by reducing the volume of a left atrium. In practicing the subject methods, the configuration of an atrium is modified or changed at least partially without the use of an implant, e.g., through chemical modification and/or application of energy to atrial tissue, where representative energy sources include RF, microwave, laser, ultrasound, cryoablative energy sources, etc. In certain embodiments, the desired atrial configuration modification is achieved by reduction of the atrial volume, e.g., through reduction of the volume of, or constricting/closing the entrance to, the atrial appendage thereof, in a manner sufficient to reduce the volume of the atrium. In certain embodiments, a catheter device comprising an RF source is employed to modulate atrial configuration according to the subject methods. Also provided are devices, systems and kits for use in practicing the subject methods. The subject methods, devices, systems and kits find use in a variety of applications, including reducing the risk of stroke in a subject suffering from atrial fibrillation.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, representative methods, devices and materials are now described.

All publications mentioned herein are incorporated herein by reference for the purpose of describing and disclosing the components that are described in the publications which might be used in connection with the presently described invention.

Methods

As summarized above, the subject invention provides methods of modulating atrial configuration, e.g., of the left and/or right atrium of a heart. By modulating atrial configuration is meant a change in the structure of the atrium. The change or modification may be any of a number of specific types of changes, including a smoothing of the interior surface of the atrium, e.g., smoothing of the tuberculated surface of the atrial appendage, a constriction or elimination of passageways (such as an atrial appendage orifice or ostium, reducing atrial volume of a heart, etc).

In certain embodiments, the atrial configuration modulation includes a reduction in atrial volume. In certain embodiments, the subject invention provides methods of reducing the volume of the left atrium of a heart, in many embodiments so that the left atrium becomes more like a conduit. The magnitude of atrial volume reduction that is achieved by practicing the subject methods is at least about 1%, sometimes at least about 5% and sometimes at least about 10% as compared to the volume of the initial atrial prior to practice of the subject methods.

A feature of the subject invention is that the atrial configuration modulation is achieved at least partially without the use of an implant. By at least partially without the use of an implant it is meant that at least some of the atrial configuration modulation that is achieved with the subject methods is achieved without the use of a structure that is delivered to and left in the atria. Although in certain embodiments the subject methods may include the use of an implant, even in such methods, at least part of the atrial configuration modulation is achieved without the use of an implant.

In certain embodiments, the atrial configuration modulation is achieved by use of chemical ablation of atrial tissue. In these embodiments, any convenient chemical ablation agent may be employed, where representative agents include, but are not limited to: organic ablation agents, such as phenol, ethanol, etc., and the like.

In certain embodiments, the atrial configuration modulation is achieved through application of energy to tissue or structures of the atria being modified. Representative types of energy that may be employed include, but are not limited to: microwave energy, laser energy, sonic energy, e.g., ultrasound, infrasound, cryoablation energy, RF energy, and the like.

In certain embodiments, RF energy is employed to achieve the above-described atrial configuration modulation, e.g., reduction in atrial volume. RF energy is well known to those of skill in the art, being employed for a variety of different medical applications. In these embodiments, the RF energy is applied to the atrium in a manner sufficient to achieve the above described atrial configuration modulation, e.g., atrial volume reduction. Any convenient RF application protocol may be employed.

In certain embodiments, the RF energy is applied to the atrial appendage of the left atrium in a manner that reduces left atrial volume. More specifically, the RF energy is applied to the left atrial appendage in a manner that at least reduces the volume of the left atrial appendage so that the overall volume of the left atrium is reduced as described above. The left atrial appendage may be reconfigured so that its volume is reduced but not eliminated, or the left atrial appendage may be substantially closed off, e.g., at the entrance or ostium, so that its volume is completely eliminated.

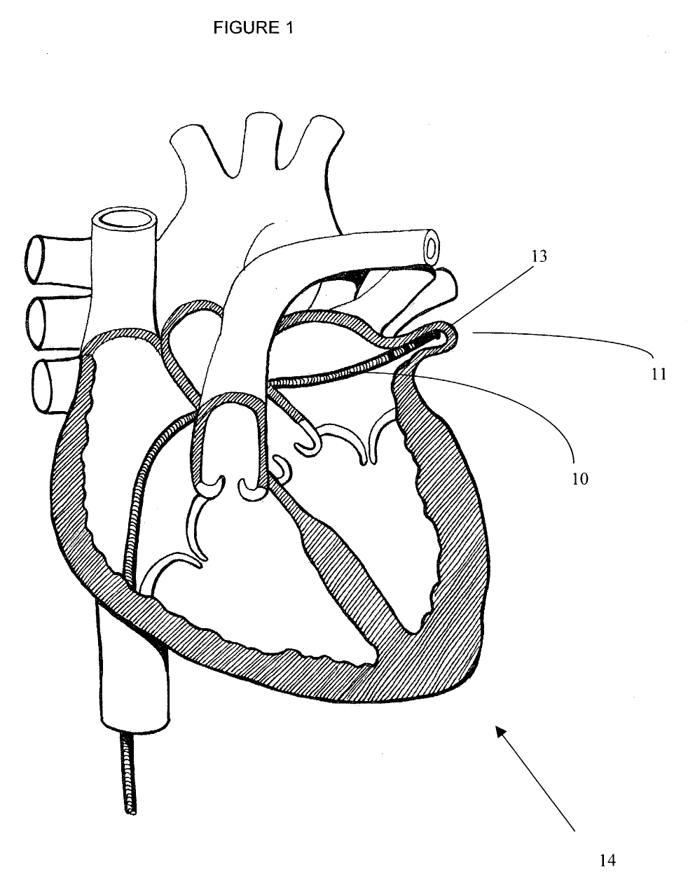

In certain embodiments, an RF catheter device, such as the one depicted in FIG. 1, is employed to reduce the volume of the left atrial appendage. In FIG. 1, catheter device 10 has distal end 13 positioned inside of the left atrial appendage 11 of heart 14. The catheter device employed may be an RF catheter device analogous to those currently available to those of skill in the art. Representative known RF catheter devices of interest include those described in U.S. Pat. Nos. 5,653,692; 6,106,520; 6,120,499; 6,322,584; and 6,358,246; the disclosures of which are herein incorporated by reference.

In using such devices, the left atrial appendage may be accessed through any of a variety of pathways as will be apparent to those of skill in the art. Trans-septal access, as depicted by FIG. 1, may be achieved by introducing a trans-septal catheter through the femoral or jugular vein, and transluminally advancing the catheter into the right atrium. Once in the right atrium, a long hollow needle with a preformed curve and a sharpened distal tip is forcibly inserted through the fossa ovalis. A radiopaque contrast media may then be injected through the needle to allow visualization and ensure placement of the needle in the left atrium, as opposed to being in the pericardial space, aorta, or other undesired location. Imaging approaches for visualizing the position of the device may be employed, e.g., transesophogeal ultrasound imaging.

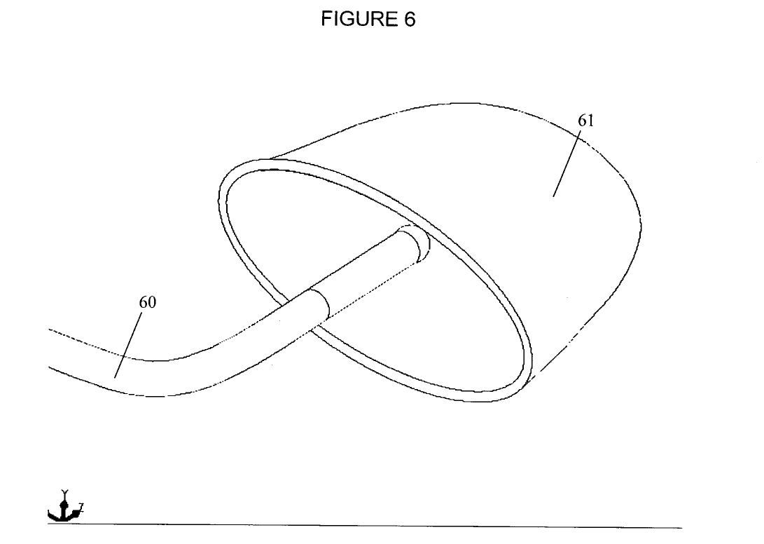

Once the position of the needle in the left atrium is confirmed, the trans-septal catheter is advanced into the left atrium. The RF catheter may then be advanced through the transeptal catheter, and steered or directed into the left atrial appendage. FIG. 6 shows a schematic view of a patient's left atrial appendage 61 with an embodiment having features of the invention 60, where the embodiment is disposed to the appendage 61 from within the heart, usually through a trans-septal approach, as described above.

Alternative approaches include venous transatrial approaches such as transvascular advancement through the aorta and the mitral valve. Other approaches of interest include, but are not limited to: transthoracic, trans pericardial, application of energy outside of the appendage (i.e., to the outer surface of the appendage), etc. In addition, the devices of the present invention can be readily adapted for use in an open-heart surgical procedure, although transluminal access is presently preferred.

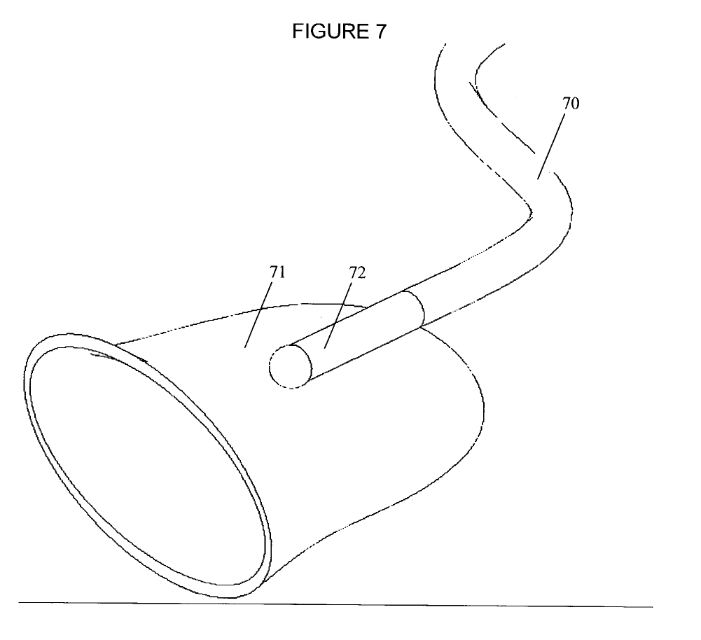

For example, a transpericardial approach may be employed in certain embodiments. FIG. 7 shows a schematic view of a patient's left atrial appendage 71 with an embodiment having features of the invention 70, where the distal transducer end 72 of the embodiment is positioned on the outside of the appendage. The access to the outside of the appendage may be obtained in an open-chest surgical procedure, but, more often, will be obtained by a transthoracical/transpericardial access way.

Devices

Also provided are devices for use in practicing the above described methods. As indicated above, any convenient device capable of achieving the desired atrial configuration modulation may be employed. In general, the devices of many embodiments at least include an energy source or sources at a distal end of an elongated structure, e.g., a catheter. The devices may further include occlusion elements, e.g., balloons, one or more lumens, e.g., for delivery of fluid to and/or removing fluid from a target tissue site (e.g., where it is desired to purge a target appendate with a non-thombogenic fluid before, during and/or after modulation of tissue), filter or shield elements, and the like. Specific representative embodiments of suitable devices for practicing the subject methods are now reviewed in greater detail.

In certain embodiments, the employed device includes a debris capture element at its distal end. A variety of different debris capture elements are of interest, including, but not limited to: a mechanically-deployed filtration element which captures any clot or particulate matter during a procedure; a suction device which pulls all surrounding fluids into the device and out of the body during the entire procedure or during short bursts of applied energy; a bag type element; a fine mesh structure to allow certain sized particles to pass; a non-porous structure for capturing all released material; and the like.

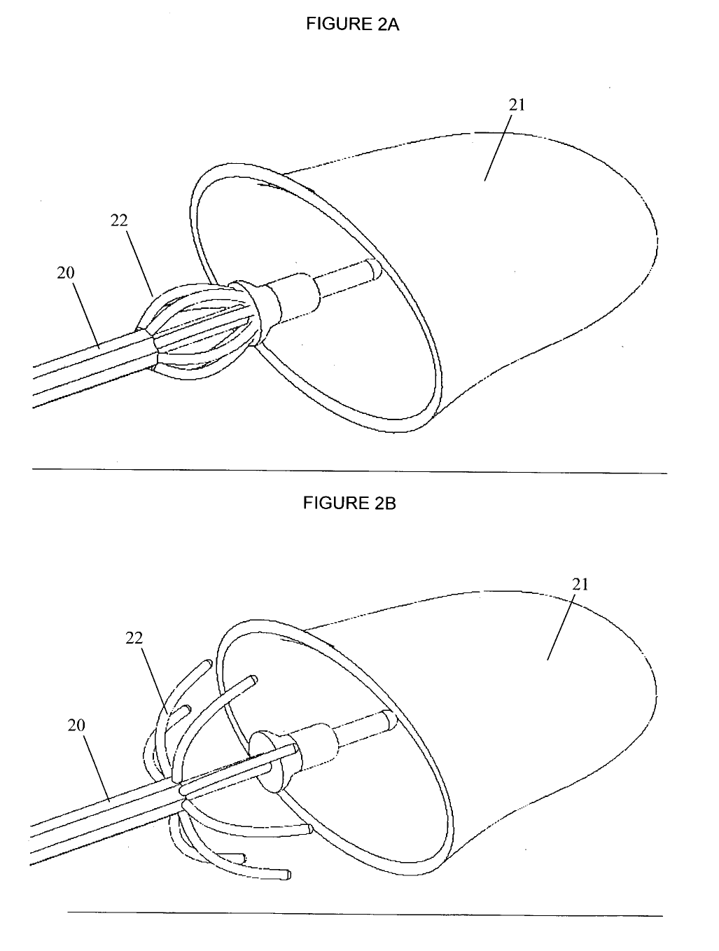

One representative device that includes a debris capture element is shown in FIGS. 2A and 2B. FIG. 2A shows the device with the debris capture element in a non-deployed state. In FIG. 2A, device 20 is shown with its distal end positioned inside of atrial appendage 21. Device 20 includes deployable filtration element 22, shown in FIG. 2A in a non-deployed position. FIG. 2B shows the same device where filtration element 22 is depicted in a deployed configuration, where in the deployed configuration the filtration element captures any debris, e.g., loose tissue or particular matter released during application of RF energy to the appendage.

In certain embodiments, the subject devices include a shielding element, e.g., for shielding non-target tissue from any energy or chemicals, e.g., RF energy, applied to the target tissue. A shielding element may have a variety of configurations, including, but not limited to: an inflatable structure that covers the appendage opening or ostium; mechanically-deployed structures; metallic structures (as for EMI shielding); optical focusing or shielding components (such as could be implemented for the case of application of laser energy for tissue configuration); a temporarily implantable shielding structure (e.g., which is then removed upon completion of the procedure, or absorbed by surrounding tissue); a structure which follows the energy transducer or could be an independent, released device (where a released device would shield an absolute position, whereas a tag-along device would shield tissue relative to the transducer location); and the like.

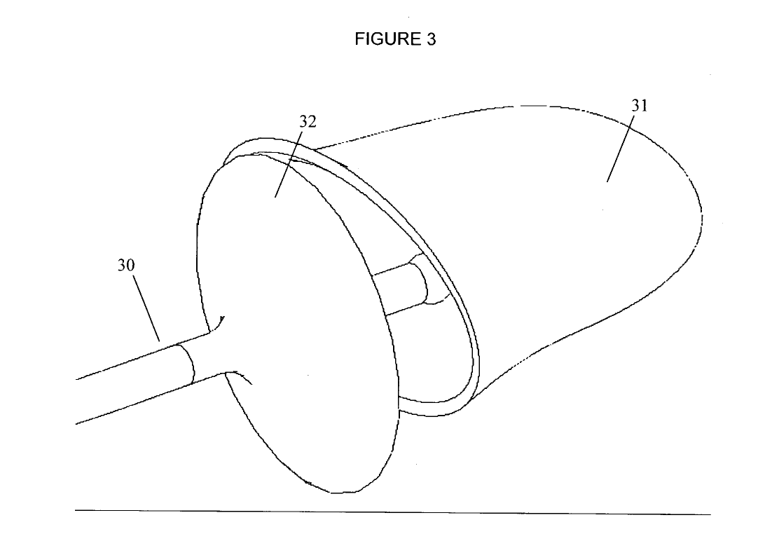

FIG. 3 shows a representative embodiment of a device that includes a shielding element. In the device shown in FIG. 3, catheter 30 has its distal end disposed in the left atrial appendage 31. The embodiment has an inflatable shield structure 32 to the distal end of the catheter, which shields ambient tissue and blood from the desired energy application, thereby reducing potential undesired effects on the blood and the tissue. The size and shape of this feature 32 could be varied through different tips, materials, material wall thicknesses, and inflation pressures.

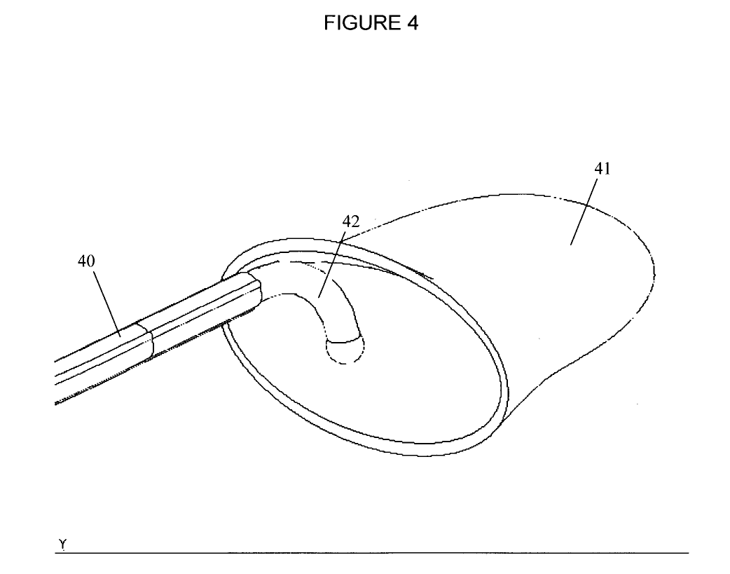

In certain embodiments, the device has a distal end configured for proper positioning of the tip at the target site, e.g., the left atrial appendage. For example, FIG. 4 shows a schematic view of a patient's left atrial appendage 41 with an embodiment 40 having features of the invention disposed in it, where the embodiment has a distal transducer tip 42 that facilitates polar positioning of the tip. The tip can be positioned radially, axially, as well as rotationally, thus providing 3-dimensional polar positioning of the energy source. This includes one rotational and two linear degrees of freedom. Other configurations include Cartesian positioning, with three linear degrees of travel.

In certain embodiments, the device is specifically configured for atrial appendage reshaping. In a representative device of this embodiment, the device includes a primary electrode mounted on an occlusion balloon and a counter electrode extending to the distal end of the device and therefore appendage. Also present is a lumen for delivery of a non-conductive media into the appendage during use. An energy source, e.g., an RF source, is in operational contact. Also present is a compliant ballon for sealing the ostium of the appendage while energy is applied during use. In certain embodiments, also present may be a lumen for the purpose of evacuating the appendage lumen (pumping out blood and or/clot). Also present may be a lumen for the purpose of introducing an RF curing agent that solidifies with the application of RF energy.

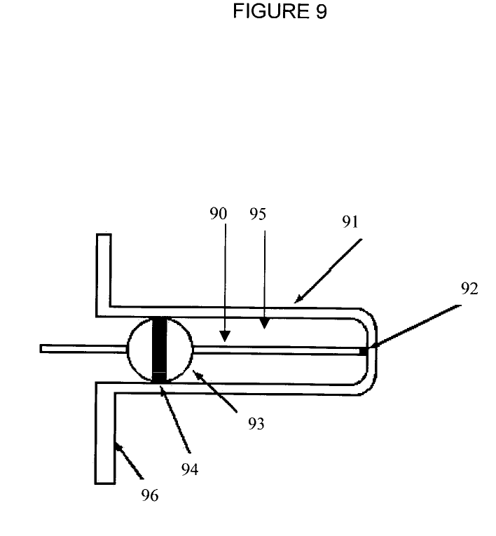

FIG. 9 shows an embodiment of the distal end of a steerable catheter 90 according to this embodiment of the subject invention. The figure schematically shows the catheter 90 disposed within the interior 95 of the atrial appendage 91. In this design, the RF cathode 92 is included in the distal tip of the catheter, and the anode 94 is placed on an inflatable occlusion balloon 93. Also shown is the atrial wall 96.

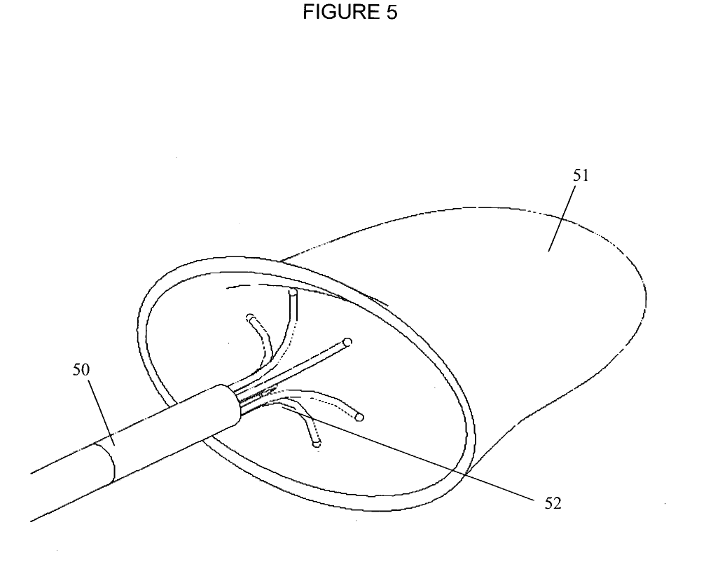

In certain embodiments, the distal end of device includes a plurality of distinct point sources of energy. By plurality is meant at least 2, where the number may be at least about 3, at least about 4, at least about 5, at least about 10 or more, depending on the desired configuration. A representative embodiment of such a device is shown in FIG. 5. FIG. 5 shows a schematic view of a patient's left atrial appendage 51 with an embodiment 50 having features of the invention disposed in it, where the embodiment has a splay tip element 52 at the distal end of the catheter. The splay tip element 52 shown here has a design with multiple energy sources for even or controlled modulation of tissue configuration. The embodiment shown depicts multiple point sources of energy. These could be individual transducers or multiple termination points from a single energy source. These tips can be controlled together or independently to map, e.g., conform, to the desired tissue topography.

Other configurations of these types of embodiments where the distal end has a plurality of distinct energy sources include line or surface energy sources. These lines or surfaces can be linear and planar or be configured in multiple axes. Energy can be varied to individual point, line, or surface energy sources. Energy emission can also be controlled and varied along a single line or surface for optimal tissue reconfiguration.

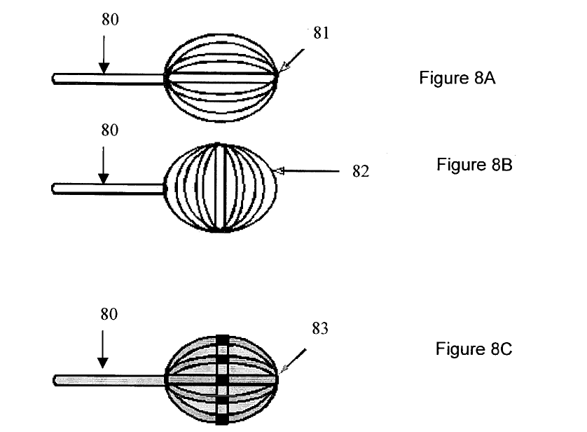

In another representative embodiment of a device having multiple energy sources at its distal end, the device includes a compliant balloon having disposed on the surface thereof multiple energy sources, such as electrodes or electrode assemblies, e.g., in the form of arrayed multiple energy sources, e.g., electrodes. In certain embodiments, the compliant balloon is made of both conductive and non-conductive materials such that the balloon itself acts as the multiple energy source, e.g., electrode, array. The multiple energy sources, e.g., electrodes or electrode assemblies, may be configured on the balloon in any convenient format, e.g., longitudinally or laterally along the balloon. FIGS. 8A and 8B show the longitudinal and lateral configurations, respectively. In FIG. 8A, distal end of catheter 80 includes includes longitudinal electrode array 81. In FIG. 8B, distal end of catheter 80 includes lateral or horizontal electrode array 82.

In certain embodiments, the multiple energy sources are electrodes covered with an insulating lubricious sheath that slides over the exterior of the electrodes, presenting an exact window of electrical contact for the appendage-electrode interface during use, where the window may be moved distal to proximal or vice versa during use. Such a device is shown in FIG. 8C, wherein device 80 includes an electrode array covered by a sheath 93.

In certain embodiments, the multiple energy sources are capacitor energy sources. An example of such a capacitor energy source is an electrode assembly that is made up of an electrode disposed within an expandable cavity of a non porous, non-conductive material, where the cavity (at least during use) includes a conductive fluid medium, e.g., a hypertonic solution, such as a salt solution, e.g., sodium chloride, etc., where the solution may be saturated with respect to the salt component, e.g., a 9% weight by volume NaCl solution. For example, electrode assembly may include a nonporous wall having an exterior for contacting tissue, with the exterior peripherally surrounding an interior area. The wall is essentially free of electrically conductive material and is adapted to assume an expanded geometry having a first maximum diameter and a collapsed geometry having a second maximum diameter, where the second diameter is less than the first maximum diameter. Also present is a lumen to convey a conductive fluid, e.g., a medium containing ions or ionic medium, into the interior area. Disposed or positioned within the area is an element, e.g., electrode, free of physical contact with the wall, where the element couples the medium within the interior area to a source of electrical energy for capacitive coupling to tissue contacting the exterior wall during use.

The electrode elements of the above-described embodiments may be fabricated from any convenient conductive material, where representative materials of interest include, but are not limited to: conductive metals, e.g., gold, platinum, iridium or combinations thereof.

As indicated above, the multiple energy sources may be selectively actuatable so that one can selectively activate each energy source according to a desired sequence or pattern, as may be indicated depending on the particular application in which the device is employed. For example, the multiple energy sources may be in operation contact with a selectable bus to drive the electrodes in a variety of modes, including, but not limited to: serial; parallel; and patterned; etc. In such embodiments, the electrodes may be in contact with a pattern generator that drives the electrodes.

As indicated above, the subject methods may be employed in conjunction with other methods of changing or modifying the atrium. For example, the subject methods may be employed to achieve a desired atrial configuration modulation prior to introduction of an atrial implant, e.g., as described in U.S. Pat. Nos. 5,989,284; 6,071,277; 6,152,144; 6,231,561; and 6,328,727; as well as in U.S. patent application Nos. 20010014800; 20020035374; and 20020049457; the disclosures of which are herein incorporated by reference.

Utility

The subject methods and devices find use in the treatment of subjects suffering from atrial fibrillation. By treatment is meant at least an amelioration of the symptoms associated with the atrial fibrillation condition afflicting the subject/patient/host, where amelioration is used in a broad sense to refer to at least a reduction in the magnitude of a parameter, e.g. symptom, associated with the pathological condition being treated. As such, treatment also includes situations where the condition, or at least symptoms associated therewith, are completely inhibited, e.g., prevented from happening, or stopped, e.g., terminated, such that the host no longer suffers from the pathological condition, or at least the symptoms that characterize the pathological condition. In certain embodiments, the subject methods reduce the risk that a subject will suffer a stroke. The magnitude of risk reduction is at least about 5%, usually at least about 10% and more usually at least about 15%, as determined using any convenient protocol.

As such, the subject methods and devices find use in the applications described in U.S. Pat. Nos. 5,989,284; 6,071,277; 6,152,144; 6,231,561; and 6,328;727; as well as in U.S. patent application Nos. 20010014800; 20020035374; and 20020049457; the disclosures of which are herein incorporated by reference.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Systems

Also provided are systems for use in practicing the subject methods, where the systems include a device, such as the representative devices described above, any may optionally include one or more additional components that find use in practicing the subject methods, e.g., conductive medium sources, energy (such as RF sources), and the like.

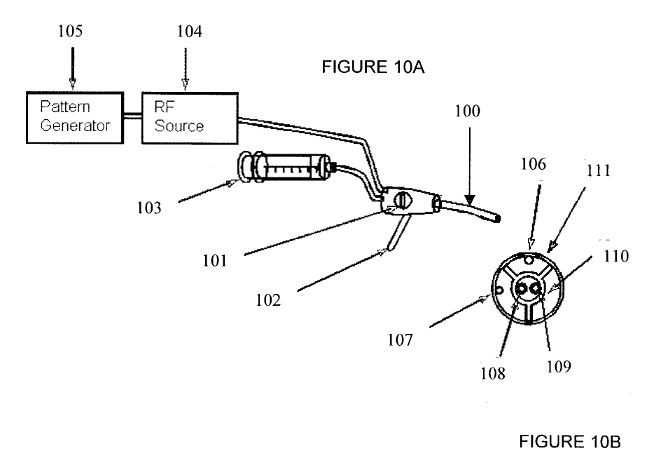

A representative system is depicted in FIGS. 10A and 10B. FIG. 10A shows an embodiment of a system having the features of the invention. This system comprises a steerable catheter 100, a first control 101 and second control 102 for the steerable distal end of the catheter, an endoflation syringe 103 for a possible distal balloon or electrode balloon on the catheter end, a radio frequency (RF) energy source 104, and a digital or analog pattern generator 105. FIG. 10B shows a cross-sectional view of the inside of the catheter, with pull-wire for steerable control 1 106 and pull-wire for steerable control 2 107, an anode conductor 108 and a cathode conductor 109, an endoflation lumen 110, and a catheter body multi-lumen extrusion 111.

Kits

Also provided by the subject invention are kits for use in practicing the subject methods. In certain embodiments, the kits of the subject invention include at least an RF device, such as an RF catheter device, as described above. In other embodiments, the kits may also include necessary supplies for the procedure. In addition, the subject kits also include instructions for practicing the above-described methods. Specifically, the subject kits also include instructions for using the RF devices of the kit in methods of reducing atrial volume. The instructions for practicing the above described methods or variations thereof are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc.

The following examples are offered by way of illustration and not by way of limitation.

### Experimental

The subject procedure closes off the left atrial appendage, which is prone to clot formation, and/or reshapes the atrial walls using catheter-based RF energy, thereby reducing the atrial volume and/or remodeling the atrial configuration. Using this procedure, a significant decrease in the likelihood of clot formation in the left atrium is accomplished. The reduction of the atrial volume induces a more constant blood flow through the atrium and leaves less space for the blood to stagnate, which in turn prevents clot formation. A feature of the methods is that tissue shrinking of the appendage need not require any deployment of a mechanical or artificial device in the atrium.

An RF catheter according to the subject invention is shown in FIG. 1. The catheter shares many characteristics with latest catheter ablation devices. Access to the left atrium is typically achieved via a Mullin's trans-septal approach whereby a trans-septal catheter and needle are delivered percutaneously from a point of insertion into the right femoral vein under local anesthesia. Single or biplanar fluoroscopy can be used to image the trans-septal catheter during the procedure and guide the distal end of the catheter to the desired site. It is therefore advantageous for at least portions of the trans-septal catheter and the RF catheter to be at least partially radiopaque. The trans-septal catheter is advanced through the right femoral vein into the right atrium and positioned adjacent to the coronary septum. The needle is advanced from the distal end of the catheter and punctures the septum in a desired location. The trans-septal catheter is then advanced over the needle through the septum and into the left atrium. Once the opening-in the interatrial septum has been created, the catheter is guided through this opening and into the left atrium. From there, the tip of the catheter is directly advanced into the area of the left atrial appendage to perform the intended tissue shrinkage to close off the appendage. To achieve the desired results, the catheter is moved to different areas of the atrial walls, primarily in the region of the atrial appendage, but not limited to it, to perform a “scoring” pattern with RF to allow for tissue shrinkage and overall volume reduction of the atrium and atrial appendage.

The catheter facilitates manipulation of the distal tip so that the RF electrode on the tip is positioned against the tissue region that is to be shrunk. Furthermore, the catheter is of sufficient flexibility to follow the pathway of the major blood vessels (e.g., V. cava) into the heart, and permits user manipulation of the tip even when the catheter is in a curved and twisted configuration. Preferably, the catheter has a distal tip portion with angulation of up to about 40°, preferably about 10° to about 30° with respect to a longitudinal axis of the catheter disposed immediately proximal to the angled distal tip portion. This angulation facilitates access to the opening of a patient's atrial appendage and appropriate positioning of the catheter's RF electrodes on or adjacent to the atrial wall. Because of the high degree of precision required for proper positioning of the RF electrode in the appendage or on the atrial wall, the catheter allows manipulation with a high degree of sensitivity and controllability as well as feedback to the user regarding the position of the catheter tip.

In another aspect of the invention, a similarly configured device is advanced transthoracically and transpericardially onto the exterior of the atrium, and is positioned on the exterior surface of the atrial appendage. Focused application of RF energy to the appendage tissue facilitates the desired tissue shrinkage and overall volume reduction of the atrial appendage.

Each of the process described above results in a sufficient reduction in atrial volume or a change in the atrial configuration so as to at least reduce the risk of clot formation in the atrium, and consequently reducing the risk of stroke in the patient.

The above results and discussion demonstrate that the present invention provides an important new and minimally invasive way to reduce and/or prevent stroke occurrence, e.g., as may arise from the presence of atrial fibrillation. Because the subject invention is a minimally invasive procedure, it provides a number of advantages over other more invasive protocols, including better patient outcome and more efficient resource use. Furthermore, no permanent implants need be deployed. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of changing the configuration of an atrium, said method comprising:
modulating tissue of said atrium at least partially without the use of an implant so that said configuration of said atrium is changed.

2. The method according to claim 1, wherein said method comprises reducing the volume of said atrium.

3. The method according to claim 1, wherein said method comprises changing the shape of said atrium.

4. The method according to claim 1, wherein said method comprises altering the tissue characteristics of said atrium.

5. The method according to claim 2, where said atrium is a left atrium.

6. The method according to claim 2, wherein said volume is reduced by applying energy to said left atrium in a manner sufficient to reduce the volume of said left atrium.

7. The method according to claim 6, wherein said energy is RF energy.

8. The method according to claim 7, wherein said RF energy is applied to said left atrium's atrial appendage.

9. The method according to claim 8, wherein said RF energy is applied to said atrial appendage in a manner sufficient to reduce said atrial appendage's volume.

10. The method according to claim 9, wherein said RF energy is applied to said atrial appendage in a manner sufficient to eliminate said atrial appendage's volume.

11. The method according to claim 8, wherein said RF energy is applied by introducing an RF source into said appendage and applying said RF energy from said introduced RF source.

12. The method according to claim 8, wherein said RF energy is applied by advancing an RF source transthoracically and transpericardially onto the exterior of said appendage and applying said RF energy from said introduced RF source externally to said appendage.

13. The method according to claim 12, wherein said source is a catheter device.

14. The method according to claim 11, wherein said source is a catheter device.

15. The method according to claim 14, wherein said catheter device is introduced percutaneously.

16. The method according to claim 15, wherein said catheter device is introduced trans-septally.

17. The method according to claim 1, wherein said method is a method of reducing a subject's risk of having a stroke.

18. A device configured to practice the method of claim 1.

19. The device according to claim 18, wherein said device is a percutaneous device.

20. The device according to claim 19, wherein said device is a catheter device.

21. The device according to claim 20, wherein said device includes an energy application element for applying energy to tissue.

22. The device according to claim 21, wherein said energy is RF energy.

23. The device according to claim 22, wherein said device includes a thermal feedback element to provide for control of thermal energy applied to atrial tissue.

24. The device according to claim 18, wherein said device includes an element for shielding ambient blood from thermal energy originating from said device.

25. The device according to claim 18, wherein said device comprises multiple energy sources at a distal end.

26. The device according to claim 25, wherein said multiple energy sources are arrayed on a surface of a compliant balloon.

27. The device according to claim 25, wherein said multiple energy sources are electrodes.

28. The device according to claim 25, wherein said multiple energy sources are capacitive energy sources.

29. The device according to claim 28, wherein said capacitive energy sources comprises an electrode assembly made up of an electrode disposed within an enclosed expandable member of a non-conductive material that is filled with a conductive medium.

30. The device according to claim 25, wherein said multiple energy sources are selectively actuatable.

31. A system for changing the configuration of an atrium, said system comprising
(a) a device according to claim 18; and
(b) a generator of an energy source.

32. The system according to claim 31, wherein said device is a device according to claim 25.

33. The system according to claim 32, wherein said system further comprises a selectable bus to drive said multiple energy sources.

34. The system according to claim 33, wherein said system further comprises a pattern generator.

35. A kit comprising:
a device according to claim 18; and
instructions for practicing the method according to claim 1.
